(19) United States
(12) Patent Application Publication
(10) Pub. No.: US 2009/0166172 A1
(43) Pub. Date: Jul. 2, 2009

(54) ETHANOL PLANT PROCESS

(76) Inventor: Leonard Ray Casey, Claremore, OK (US)

Correspondence Address: CORNELIUS P. DUKELOW;ABINGTON INTELLECTUAL PROPERTY LAW GROUP, PC, 10026-A S, MINGO ROAD, NO. 240, TULSA, OK, 74133, US

(21) Appl. No.: 11/966,337

(22) Filed: Dec. 28, 2007

(51) Int. Cl. C07C 29/80

(52) U.S. Cl. 203/19

(57) ABSTRACT

The present invention conserves water by reducing the heat load placed on a cooling tower during the ethanol fuel production process. An air cooler is placed between the ethanol vapor condenser and the cooling tower thereby minimizing process temperature spikes before water enters the cooling tower. This, in turn, shortens the process cycle thereby increasing ethanol production. During certain climactic conditions, the cooling tower may be completely bypassed, thereby conserving more water and increasing ethanol production.

### BACKGROUND OF THE INVENTION

1. Field of the Invention

The present invention relates to ethanol fuel plants and ethanol fuel plant processes.

2. Description of Related Art

Ethyl alcohol, the same alcohol found in alcoholic beverages, may also be used as a bio-fuel alternative to gasoline. The increasing cost of gasoline is making “ethanol fuel” a viable alternative energy source to fossil fuels and their derivatives. Because it is easy to manufacture and process, and can be made from common materials, ethanol fuel is steadily becoming a promising alternative to gasoline throughout much of the world.

Ethanol can be mass-produced by fermentation of sugar or by hydration of ethylene from petroleum and other sources. Current interest in ethanol mainly lies in bio-ethanol, produced from the starch or sugar in a wide variety of crops. There are concerns relating to large amount of arable land required for crops, as well as the energy and pollution balance of the ethanol production cycle. Recent developments with cellulosic ethanol production and commercialization may allay some of these concerns.

According to the International Energy Agency, cellulosic ethanol could allow ethanol fuels to play a much bigger role in the future than previously thought. Cellulosic ethanol can be made from plant matter composed primarily of inedible cellulose fibers that form the stems and branches of most plants. Dedicated energy crops, such as switchgrass, are also promising cellulose sources that can be produced in many regions of the United States.

Bio-ethanol is obtained from the conversion of carbon based feedstock. Agricultural feedstocks are considered renewable because they get energy from the sun using photosynthesis, provided that all minerals required for growth (such as nitrogen and phosphorus) are returned to the land. Ethanol can be produced from a variety of feedstocks such as sugar cane, bagasse, miscanthus, sugar beet, sorghum, grain sorghum, switchgrass, barley, hemp, kenaf, potatoes, sweet potatoes, cassava, sunflower, fruit, molasses, corn, stover, grain, wheat, straw, cotton, other biomass, as well as many types of cellulose waste and harvestings, whichever has the best well-to-wheel assessment.

The basic steps for large scale production of ethanol are: microbial (yeast) fermentation of sugars, distillation, dehydration, and denaturing (optional). Prior to fermentation, some crops require saccharification or hydrolysis of carbohydrates such as cellulose and starch into sugars. Enzymes convert starch into sugar.

For the ethanol to be usable as a fuel, water must be removed. Most water is removed by distillation, but purity is limited to 95-96% due to formation of a low-boiling water-ethanol azeotrope. 96% m/m (93% v/v) ethanol, 4% m/m (7% v/v) water mixture may be used as a fuel.

Currently, the most widely used purification method is a physical absorption process using a molecular sieve. Another method, azeotropic distillation, is achieved by adding the hydrocarbon benzene which also denatures the ethanol rendering it undrinkable for duty purposes. A third method involves use of calcium oxide as a desiccant.

Ethanol is most commonly used to power automobiles, though it may be used to power other vehicles, such as farm tractors and airplanes. Ethanol (E100) consumption in an engine is approximately 34% higher than that of gasoline (the energy per volume unit of ethanol is 34% lower than that of the same volume unit of gasoline). However, higher compression ratios in an ethanol-only engine allow for increased power output and better fuel economy than would be obtained with the lower compression ratio. Ethanol-only engines are generally tuned to give slightly better power and torque output to gasoline-powered engines. In flexible fuel vehicles, the lower compression ratio requires tunings that give the same output when using either gasoline or hydrated ethanol.

The top five ethanol producers in 2006 were Brazil (4.491 billion US gallons per year (bg/y)), the United States (4.855 bg/y), China (1.017 bg/y), India (0.502 bg/y) and France (0.251 bg/y). Brazil and the United States accounted for 90 percent of all ethanol production. Strong incentives, coupled with other industry development initiatives, are giving rise to fledgling ethanol industries in countries such as Thailand, the Philippines, Colombia, the Dominican Republic, and Malawi. Most cars on the road today in the U.S. can run on blends of up to 10% ethanol, and motor vehicle manufacturers already produce vehicles designed to run on much higher ethanol blends. Portland, Oregon, recently became the first city in the United States to require all gasoline sold within city limits to contain at least 10% ethanol. Several motor vehicle manufacturers, including Ford, DaimlerChrysler, and GM, sell “flexible-fuel” cars, trucks, and minivans that can use gasoline and ethanol blends ranging from pure gasoline up to 85% ethanol (E85). By mid-2006, there were approximately six million E85-compatible vehicles on U.S. roads.

### BRIEF SUMMARY OF THE INVENTION

Cooling towers are evaporative coolers used for cooling water or other working medium to near the ambient wet-bulb air temperature. Cooling towers used in the ethanol fuel production process use evaporation of water to reject heat from the process. The towers vary in size from small factory-built units to very large on-site constructed hyperboloid structures that can be up to 200 meters tall and 100 meters in diameter.

Cooling towers work by extracting heat to the atmosphere though the cooling of a water stream to a lower temperature. The type of heat rejection in a cooling tower is termed “evaporative” in that it allows a small portion of the water being cooled to evaporate into a moving air stream to provide significant cooling to the rest of that water stream. The heat from the water stream transferred to the air stream raises the air's temperature and its relative humidity to 100%, and this air is discharged to the atmosphere.

Although cooling towers serve their particular purpose, they have several drawbacks: they do not work properly at very cold temperatures, they are not water conservation friendly, they can spread pollutants over wide areas in the form of evaporated water (drift), they create an increased risk of spreading airborne pathogens, and they use large amounts of electricity to run fans.

The present invention conserves water by reducing the heat load placed on a cooling tower during the ethanol fuel production process. An air cooler is placed between the ethanol vapor condenser and the cooling tower (preferably as close to the condenser as is physically possible) thereby minimizing process temperature spikes before water enters the cooling tower. This, in turn, shortens the process cycle thereby increasing ethanol production. During certain climactic conditions, the cooling tower may be completely bypassed, thereby conserving more water and increasing ethanol production. The decreased use of water also reduces pollution drift, the risk of spreading airborne pathogens, and electricity consumption.

On average, three gallons of fresh water are used to produce one gallon of ethanol. A typical ethanol plant producing 100 million gallons of ethanol a year uses approximately 300 million gallons of fresh water per year during the ethanol production process. Furthermore, cooling towers themselves can evaporate approximately 9 gallons of water for every one gallon of ethanol produced. (An ethanol plant producing 47 million gallons of ethanol per year produces a heat load of approximately 42,000 BTU per minute. With a ΔT of approximately 20° and a conversion rate of 500, approximately 420 million BTU are produced per hour. A cooling tower uses approximately 1000 pounds of water to expel 1 million BTU resulting in the use of approximately 420 thousand pounds of water per hour or 50360 gallons of water per hour. Therefore, a plant operating for approximately 8500 hours per year uses approximately 420 million gallons of water per year to produce 47 million gallons of ethanol.) It is estimated that using an air cooled heat exchanger as described herein will reduce water consumption in a typical ethanol plant by approximately 20-25%. It is also estimated that using an air cooled heat exchanger as described herein will increase ethanol production in a typical ethanol plant by 0.5-2% due to increased efficiency of the ethanol plant.

### BRIEF DESCRIPTION OF THE SEVERAL VIEWS OF THE DRAWING

A preferred embodiment of the present invention is described in detail below with reference to the attached drawing FIGURES, wherein:

FIG. 1 illustrates a schematic flowchart of the method as described herein.

### DETAILED DESCRIPTION OF THE INVENTION

The embodiments discussed herein are merely illustrative of specific manners in which to make and use the invention and are not to be interpreted as limiting the scope of the instant invention.

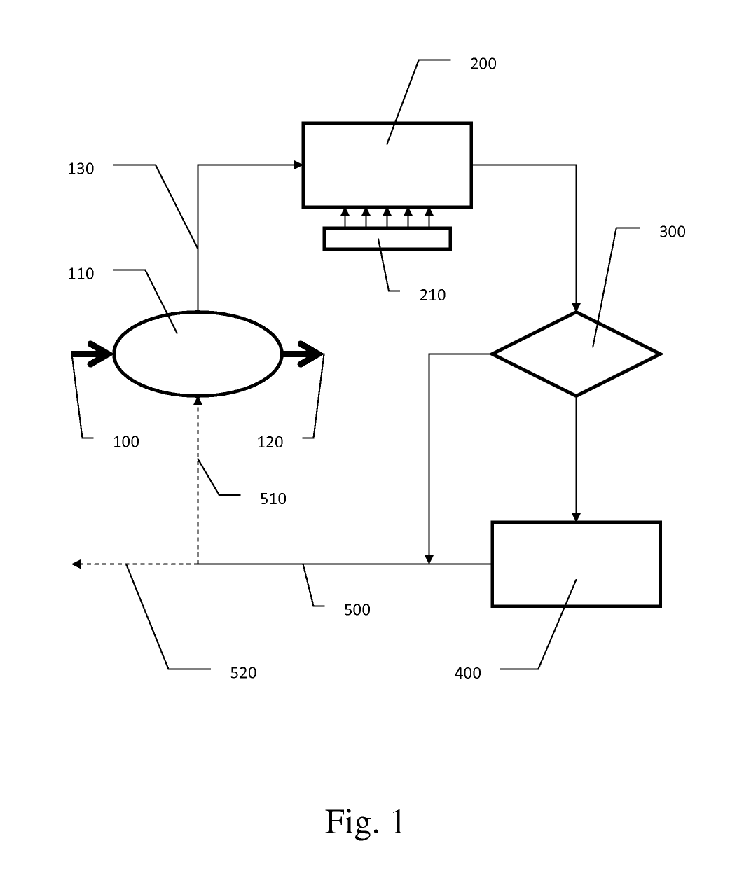

Referring to the drawing in detail, FIG. 1 illustrates a schematic flowchart of the method as described herein. Ethanol vapor from the plant process 100 is directed into a condenser 110 (shell & tube, etc.) wherein the ethanol vapor is condensed into liquid ethanol (by exchanging heat with water) and directed out of the condenser for further plant processing 120. The heated water 130 leaves the condenser and is directed through an air cooled heat exchanger 200 wherein the heated water is cooled by exchanging heat with the air. In the preferred embodiment, the air cooled heat exchanger is placed as close to the condenser as is physically possible. Air circulation over the air cooled heat exchanger may be increased by the addition of a fan or fans 210.

After passing through the air cooled heat exchanger 200, the water is directed to a control valve 300. As the water passes through the control valve, it may be directed to the cooling tower 400, or it may be directed back into the plant's water circulation system 500, thereby completely bypassing the cooling tower. After passing back into the plant's water circulation system it may be directed back for use in the condenser 510, or it may be directed for use in other plant processes 520.

While the invention has been described with a certain degree of particularity, it is to be noted that modifications may be made in the details of the invention's construction and the arrangement of its components without departing from the spirit and scope of this disclosure. It is understood that the invention is not limited to the embodiments set forth herein for the purposes of exemplification.

The invention claimed is:

1. An improved method for producing ethanol, comprising the steps of:
directing water used in the ethanol condensation process to an air cooled heat exchanger;
directing water from said air cooled heat exchanger to a cooling tower; and
directing water from said cooling tower back for use in the ethanol production process.

2. The method of claim 1, further comprising the steps of:
measuring the temperature of water after leaving said air cooled heat exchanger and before entering said cooling tower;
selectively bypassing said cooling tower when temperature of water leaving said air cooled heat exchanger meets predetermined criteria; and
directing water back for use in the ethanol production process.

3. The method of claim 2, further comprising the steps of:
measuring the temperature of water at some point after leaving said condensation process and before entering said air cooled heat exchanger;
selectively activating or deactivating fans used to increase air flow over said air cooled heat exchanger when temperature of water leaving said condensation process meets predetermined criteria.
